(19) United States
(12) Patent Application Publication
(10) Pub. No.: US 2023/0356215 A1
(43) Pub. Date: Nov. 9, 2023

(54) TEST STRIPS FOR DETERMINATION OF UREA LEVELS IN SALIVA AND FOR DETECTION OF AZOTEMIA OR KIDNEY DISEASE IN FELINE AND CANINE SUBJECTS

(71) Applicant: SN Biomedical Inc., Leduc County (CA)

(72) Inventors: Matthew Nickel, Edmonton (CA); Hilary Sweet, Leduc County (CA)

(73) Assignee: SN Biomedical Inc., Leduc County, AB (CA)

(21) Appl. No.: 18/245,680

(22) PCT Filed: Sep. 14, 2021
(86) PCT No.: PCT/CA2021/051280

§ 371 Date: Mar. 16, 2023

Related U.S. Application Data
(60) Provisional application No. 63/079,184, filed on Sep. 16, 2020.

(51) Int. Cl. B01L 3/00; C12Q 1/58; G01N 33/84; G01N 33/52

(52) U.S. Cl. CPC B01L 3/5023; C12Q 1/58; G01N 33/84; G01N 33/523; G01N 2800/347; B01L 2300/0825; B01L 2300/069; B01L 2200/16

(57) ABSTRACT

Disclosed are diagnostic test strips to screen for azotemia or kidney disease in felines and canines. The diagnostic test strips comprise an elongate carrier material to which is adhered a test pad assembly comprising a reagents substrate material loaded with urease, a pH indicator, and a buffer for adjusting a pH value of a feline or canine saliva sample to within a transition interval of the pH indicator, a laminate layer, and an adhesive layer. The test strips may additionally comprise a control pad assembly adhered to the elongate carrier material adjacent to the test pad assembly.

### TECHNICAL FIELD

This disclosure generally relates to detection of azotemia or kidney disease in feline and canine subjects. More specifically, this disclosure pertains to devices configured for detecting the levels of urea in the saliva of feline and canine subjects for use in the diagnosis of azotemia or kidney disease.

### BACKGROUND

Chronic kidney disease is very common in domesticated feline and canine populations and may affect as many as 35% of cats and 10% of dogs. In fact, some studies found kidney disease to be the most common cause of death in cats greater than 5 years of age. Thus, early detection of kidney failure is critical in both feline and canine patients so that measures may be taken to increase longevity and quality of life including, for example, changes in diet to restrict protein and phosphorus intake.

Urea is an end product in protein metabolism, and levels of urea concentration in the blood provide an indicator of kidney function. For example, end-stage kidney disease is typically associated with a reduced glomerular filtration rate (GFR) that commonly results in increased blood urea and creatinine, a condition known as azotemia. It has also been found that accompanying increases in salivary urea and creatinine occur during kidney failure in human patients. As a result, salivary urea nitrogen dipsticks and optical biosensors have been developed for human medical diagnostic applications.

The use of diagnostic test strips to detect blood urea or urea nitrogen in biological samples collected from human subjects began in the late 1960s and early 1970s. Such diagnostic test strips conventionally use the enzyme urease to hydrolyze urea into ammonia and carbon dioxide as shown in the equation (1) below:

CO(NH₂)₂+H₂O→2NH₃+CO₂[or (NH₄)₂CO₃] (1)

The hydrolysis of urea may then be detected using a pH indicator. Such diagnostic test strips however, suffered from various drawbacks including short shelf life (about one month when stored in a dark bottle with desiccants) and short color stabilization periods (color starts to fade after 2 minutes).

More recently, the salivary urea test strips made for human patients have been used for detection of elevated blood urea in canines. However, the chemical environment of canine and feline mouths varies significantly from humans.

In general, feline saliva and canine saliva have more basic pH values than human saliva. The average oral pH of canines is generally about 8.53±0.34 while the average oral pH of felines is generally about 8.6±0.7. In contrast, human saliva generally has a pH range of about 6.2 to about 7.6 with an average of about 6.7. As well, feline saliva and canine saliva have higher buffering capacity than human saliva. The main buffering component in human, feline, and canine saliva is a bicarbonate buffer system. Humans may have bicarbonate concentrations in the range of 11.6 to 17.9 mmol/L, whereas feline saliva may have bicarbonate concentrations of about 26 mmol/L. Canine saliva generally have bicarbonate concentration ranges of about 10 to 50 mmol/L. Thus, diagnostic test strips developed for use with humans do not produce accurate results for feline and canine subjects.

Further, humans and large canines produce more saliva than small canines and felines. Conventional test strips developed for human use require about 40 microliters (μL) of saliva collected by a mouth swab which is extracted from the swab by centrifugation, and then pipetted onto a test pad surface. Not only is this a complex procedure, but it also requires a volume of saliva that is not easily collected from felines or small canines. This problem is exacerbated by the occurrence of chronic kidney disease as it may cause extreme dehydration and therefore a dry mouth in the animal.

### SUMMARY

Embodiments of the present disclosure generally relate to diagnostic test strips and kits configured for the detection of azotemia or kidney disease in feline and canine subjects.

According to an example embodiment disclosed herein, a test strip for detecting azotemia or kidney disease in felines and canines may comprise an elongate carrier material to which is adhered a reagents substrate material loaded with urease, a selected pH indicator, and a selected buffer composition for adjusting a pH value of a feline or canine saliva sample to within a transition interval of the selected pH indicator.

Another example embodiment disclosed herein relates to a kit for detecting azotemia or kidney disease in felines and canines, comprising a plurality of diagnostic test strips disclosed herein, with instructions for their use.

### BRIEF DESCRIPTION OF THE FIGURES

The embodiments of the present disclosure will be described with reference to the following drawings in which:

FIG. 1 is an exploded perspective view of a test strip suitable for detecting azotemia or kidney disease in felines and canines according to one embodiment of the present disclosure;

FIG. 2 is an example of a scoring system for determining whether a feline or canine has azotemia or kidney disease for use with the diagnostic test strips disclosed herein;

FIG. 3 is a plot of salivary urea nitrogen and blood urea nitrogen in 92 canine subjects;

FIG. 4 is a plot of salivary urea nitrogen and blood urea nitrogen in 56 feline subjects;

FIG. 5 is a receiver operating characteristic curve for diagnosing elevated blood urea nitrogen (≥11.1 mM) in canine subjects using a test pad of the present disclosure alone or in conjunction with a control pad;

FIG. 6 is a receiver operating characteristic curve for diagnosing elevated blood urea nitrogen (≥12.9 mM) in feline subjects using tests pads according to the present disclosure;

FIG. 7 is a chart showing the relationship between the hue of an example test strip disclosed herein and the urea concentration of a synthetic saliva sample placed onto the test strip; and

FIG. 8 is a chart showing the effects of selected stabilizers on the % activity of a test strip over extended time periods.

### DETAILED DESCRIPTION

The embodiments of the present disclosure generally relate to diagnostic test strips configured for detection of azotemia or kidney disease in feline and canine subjects. More particularly, the embodiments of present disclosure relate to diagnostic test strips suitable for determining salivary urea nitrogen (SUN) as an indicator for detection of the occurrence of azotemia or kidney disease in feline and canine subjects. Some embodiments disclosed herein relate to kits configured for detection and detecting the occurrence of azotemia or kidney disease in feline and canine subjects.

The diagnostic test strips of the present disclosure may have a number of advantages. One advantage of the diagnostic test strips includes a reduction in false positives resulting in incorrect diagnoses of azotemia or kidney disease. In more detail, because the components of the diagnostic test strips of the present disclosure are selected specifically for the detection of azotemia or kidney disease in felines and canines, there is a significant reduction in the occurrence of false positives. In contrast, prior techniques have used diagnostic test strips configured for human use for detection of kidney disease in domesticated dogs. However, as previously described herein, the chemical environment in a human mouth is substantially different from that of a feline or a canine. Thus, while diagnostic test strips configured for human use may be functional in the typical pH ranges of a human mouth, such test strips have limited functionality in the pH ranges that are commonly associated with feline and canine mouths. As a result, diagnostic test strips prepared for use to assess human saliva, produce unreliable results by way of showing a false positive condition in a healthy animal.

Another advantage associated with the test strips disclosed herein includes a requirement for a significantly smaller saliva sample than is typically necessary of conventional techniques to produce a usable result. Some conventional techniques may require a saliva sample of about 40 μL, which, as described above, may be difficult to acquire from felines, small canines, or from felines and canines that have azotemia or kidney disease, which can result in severe dehydration. As will be described below, the diagnostic test strips of the present disclosure may only require a saliva sample of about 2 μL to about 5 μL to provide a reliable result.

Yet another advantage associated with the diagnostic test strips of the present disclosure includes significantly increased stability and shelf life as compared to conventional test strips. In more detail, the shelf life of conventional diagnostic test strips is about 1 month when stored in a desiccator in a dark environment, after which time, the enzyme activity of the strips deteriorates to a state such that the diagnostic test strips are no longer functional. In contrast, and as will be detailed below, the diagnostic test strips of the present disclosure may have shelf lives of more than 12 months when stored in sealed HDPE bottles at room temperature, even without the use of a desiccant.

Further advantages will be discussed below and will be readily apparent to those of ordinary skill in the art upon reading the present disclosure.

Reference will now be made in detail to example embodiments of the disclosure wherein numerals refer to components, examples of which are illustrated in the accompanying drawings that will further show the example embodiments without limitation.

An example of an embodiment of the present disclosure relates to a test trip for detecting azotemia or kidney disease in felines and canines comprising an elongate carrier material to which is adhered a reagents substrate material loaded with urease, a selected pH indicator, and a selected buffer component suitable for adjusting a pH value of a feline saliva sample or a canine saliva sample to within a transition interval of the selected pH indicator.

As used herein, “azotemia” refers to elevated blood urea nitrogen (BUN) and serum creatinine levels.

As used herein, “kidney disease” refers to sudden or gradual loss of kidney function in a mammalian subject.

As used herein, the term “feline” refers to members of the Felinae family. Such members include domesticated cats as well as bay cats, caracals, ocelots, lynxes, pumas, leopards, bobcats, cheetahs, cougars, and the like that may be occasionally or routinely assessed by veterinarians. In one aspect, the domesticated cats are companion animals.

As used herein, the term “canine” refers to members of the Caninae family. Such members include domesticated dogs as well as wolves, coyotes, jackals, foxes, and the like that may be occasionally or routinely assessed by veterinarians. In one aspect, the domesticated dogs are companion animals.

It is noted that, while the present disclosure generally relates to domesticated felines and domesticated canines, it should be understood that the diagnostic test strips and kits disclosed herein may be used with non-domesticated types of felines and canines for detection of azotemia or kidney disease by veterinarians.

As used herein, “test strip” refers to an elongate carrier material to which is adhered an assembly configured for receiving thereon an analyte and determining chemical constituents comprising the analyte. In the context of the present disclosure, the “analyte” is a feline or canine saliva sample. In more detail, the diagnostic test strips disclosed herein comprises an elongate carrier material to which is adhered a reagents substrate material loaded with selected compounds to enable and facilitate the detection and determination of the urea nitrogen content of a feline or canine saliva sample.

As used herein, a “reagents substrate material” is a base material for receiving thereon and therein urease, a selected pH indicator, a buffering component, and optionally one or more selected suitable additives without significantly altering the physicochemical properties of the components or altering the constituents of the saliva sample.

As used herein, the term “loaded with” means impregnated coated onto or into a reagents substrate material of the test strip. The reagents substrate material may have one or more assemblies comprising layers of urease, pH indicator, buffer component, and optionally one or more selected additives, affixed thereto. Alternatively, the urease, the pH indicator, the buffer component, and other selected additives may be separately impregnated into the reagents substrate material.

As used herein, “pH indicator” generally refers to colorimetric pH indicators that may indicate a specific target pH or target pH range by a change in color and/or a change in color intensity. Addition of a feline saliva sample or a canine saliva sample collected from an animal with azotemia or kidney disease may result in the appearance of a specific color and/or a change in color intensity associated with a target pH or pH range thereby enabling a visual detection and determination of azotemia or a kidney disease condition. Suitable pH indicators may have a “transitional interval” or pH range across which they are effective.

As used herein, an “elongate carrier material” refers to a material or substrate to which the reagents substrate material may be adhered. The elongate carrier material may be used to facilitate the handling of the test strips of the present disclosure and/or the collection of saliva samples from the felines or canines. In some aspects, the elongate carrier material may be a strip of rigid or semi-rigid polymeric material. In other aspects, the elongate carrier material may be a swab formed of wood, a paper-based material, or a plastic. In a yet further aspect, the elongate carrier material may be a disposable latex or nitrile glove. Further, in some aspects, the elongate carrier material may be a substrate that encourages the feline or canine to put the test strips of the present disclosure into their mouths such as a chew toy.

As used herein, “salivary urea nitrogen” or “SUN” refers to the amount of nitrogen in a saliva sample derived from the molecule urea.

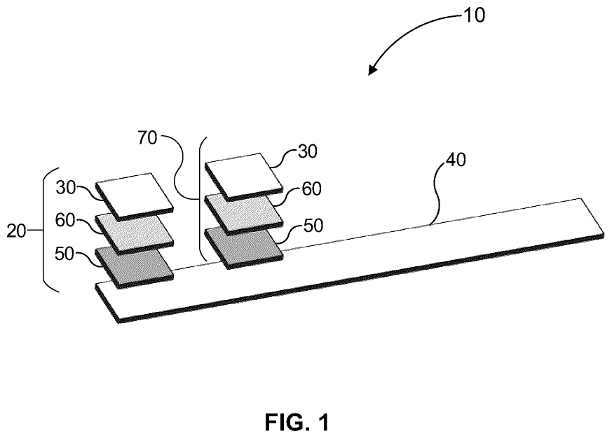

An embodiment of an example test strip 10 configured for detecting azotemia or kidney disease in saliva samples collected from felines and canines, is shown in FIG. 1. The illustrated test strip 10 comprises an elongate carrier material 40 wherein a first end of the elongate carrier material 40 is a gripping and a handling end and the opposite end of the elongate material has affixed thereto a test pad assembly 20 adjacent to but spaced apart from a control pad assembly 70.

In this example test strip 10, each of the test pad assembly 20 and the control pad assembly comprises three layers 30, 50, 60 wherein the uppermost layer 30 is a reagents layer, the bottom layer 50 is an adhesive layer, and the middle layer 60 is a laminate layer.

The reagents layer 30 of the test pad assembly 20 comprises a substrate that is loaded with urease, a pH indicator, and a buffer component. When contacted with a saliva sample, the reagents layer 30 may react with the saliva sample to produce a color and/or a color change. According to one aspect, the reagents layer 30 may be separately coated with each of the urease, the pH indicator, and the buffer component. According to another aspect, the reagents layer 30 may be impregnated with the urease, the pH indicator, and the buffer component. The impregnation may be performed by soaking the reagents layer 30 in a solution comprising the urease, the pH indicator, and the buffer component such that each of the reagents are adsorbed onto and absorbed with the substrate. In some aspects, the substrate of the reagents layer 30 comprises an absorbent material such as cellulose, a cellulose derivative, a polyester, a nylon, and the like. In some aspects, the cellulose derivative may be a nitrocellulose or a cellulose acetate. According to a particular aspect, the substrate of the reagents layer 30 may be a cotton linter filter paper such as a Grade CF1 provided by Cytiva Life Sciences. The substrate of the reagents layer 30 may have a thickness of about 150 μm to about 200 μm. In a further aspect, the reagents layer 30 may have a thickness of about 175 μm. As a result of the thickness and water absorptivity properties of the reagents layer 30, there may only be required a minimal amount of sample to fully wet the reagents layer 30. In some aspects, the reagents layer 30 may have a surface area of about 0.10 cm² to about 0.40 cm². In a further aspect, the reagents layer 30 may have a surface area of about 0.15 cm². Thus, in such aspects, there may only be required about 2 μL to about 5 μL of saliva to completely wet the reagents layer 30. As a result, the test strip 10 of the present disclosure may advantageously be well suited to testing saliva samples of small felines and small canines, which may produce less saliva than their larger family members, or those that have extreme dehydration due to azotemia or kidney disease, as previously described herein. Thus, the reagents layer 30 may be configured to be sufficiently water permeable to receive and maintain the saliva therein to facilitate the reaction with the urease, the pH indicator, and the buffer. In some aspects, the reagents layer 30 has a water absorption rate of about 15 μL/cm² to about 30 μL/cm². In a further aspect, the reagents layer 30 has a water absorption rate of about 18 μL/cm² to about 25 μL/cm².

As disclosed above, the reagents layer 30 is loaded with urease. The urease may be any commercially available urease, for example, such as those sourced from canavalia ensiformis (jack bean), watermelon seeds, pea seeds, and the like. Further, the urease may be loaded in a concentration sufficient for hydrolyzing the urea present in a saliva sample to produce ammonia (NH₃) and carbon dioxide (CO₂) or alternatively, ammonium carbonate ((NH₄)₂CO₃) to thereby affect a change in pH. Thus, in some aspects, the urease may be loaded in a concentration greater than 100,000 IU/L, wherein one IU is the amount of enzyme causing the liberation of 1 μmol ammonia per minute at 25° C. and pH 7. In a particular aspect, the urease may be loaded in a concentration of about 540,000 IU/L.

The reagents layer 30 is also loaded with a pH indicator. As discussed above, the hydrolyzation of urea to NH₃ and CO₂ ((NH₄)₂CO₃) causes an increase in pH. Thus, the pH indicator is selected such that the increase in pH caused by the hydrolyzation of urea causes the pH indicator to change color as shown below by equation (2):

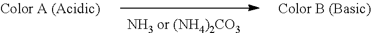

In more detail, the pH indicator is selected such that it detects and indicates pH changes over the effective range of urease, which corresponds to a pH of about 5 to about 9. In some aspects, the pH indicator may be selected such that it detects and indicates pH changes over a pH range of about 6 to about 8. Suitable pH indicators include phenol red, bromothymol blue, bromocresol purple, methyl red and the like. Further, it was found that pH indicators that are sparingly soluble in water may be beneficial, as such indicators may reduce leaching of the color from the test strip when wetted. It is noted that, in the context of the present disclosure, “sparingly soluble” means a solubility of about 1 g/100 mL to about 3.5 g/100 mL. In such aspects, bromothymol blue has been found to be useful. According to one aspect, the pH indicator may be present in a concentration of about 1 mM to about 5 mM. In a particular aspect, the pH indicator is present in a concentration of about 2 mM.

The reagents layer 30 is also loaded with a buffer component. As indicated above, the buffer component is included to adjust an initial pH value of a feline or canine saliva sample to within the transition interval of the selected pH indicator. Generally, it has been found that buffers that adjust the initial pH of the saliva sample to about the lowest pH of the pH indicator's transition interval are useful, as they facilitate significant changes in color of the pH indicator over its full transition interval. The buffer components must have a suitable buffering capacity over the pH range of the indicator as well as the pH range of feline and canine saliva. Buffers with at least one pKa (acid dissociation constant) near that of the pH indicator and a buffering capacity from about pH 6 to about pH 9 are optimal. The buffer components also serve a number of additional purposes such as providing an environment suitable for urease functionality and stability. In more detail, as discussed above, urease is functional in pH ranges of about 5 to about 9, and, as a result, the buffer may be selected such that it maintains that pH range.

In light of the above, non-limiting examples of buffers suitable for use in the test strip 10 of the present disclosure include sodium phosphate, potassium phosphate, 2-[Bis(2-hydroxyethyl)amino]-2-(hydroxymethyl)propane-1,3-diol (Bis-tris methane), and 2,2′-(Propane-1,3-diyldiimino)bis[2-hydroxymethyl)propane-1,3-diol] (Bis-tris propane). Further, in some aspects, the buffer is present in a concentration of about 1 mM to about 100 mM. In a particular aspect, the buffer is present in a concentration of about 40 mM. In a particular aspect, the buffer to be used is dependent on the buffering capacity of the composition of the test material and can be adjusted by one skilled in the art of making chemical diagnostic tests.

Further, in some embodiments, the reagents layer 30 may be loaded with one or more additional additives. Suitable additives include wetting agents, thickening agents, stabilizers, sensitizing agents, and the like.

In one aspect, the reagents layer 30 is further loaded with one or more wetting agents. Wetting agents may be used to improve the homogeneity of wetting throughout the reagents layer 30 to thereby improve uniformity of the color provided by the pH indicator. In a further aspect, the wetting agent may comprise a polyethylene glycol, a polysorbate, or a combination thereof. In a particular aspect, the polyethylene glycol may have a molecular weight of about 3500 to about 4500. In another particular aspect, the polysorbate is polysorbate 80.

In a further aspect, the reagents layer 30 may be further loaded with one or more stabilizers to improve the shelf life of the test strips 10. In one aspect, the one or more stabilizers may comprise a serum albumin, a saccharide, or a combination thereof. In general, serum albumins may be used as stabilizers due to their ability to reduce protein surface adsorption, to increase the glass transition temperature of dried protein products, and to provide general stabilization of proteins during drying processes. Selected saccharides may be used as stabilizers due to their ability to form hydrogen bonds with protein molecules, thereby acting as water-replacement molecules during drying and maintaining the shape and thus functionality of the protein, and due to their ability to increase the glass transition temperature of dried protein products, thereby reducing protein mobility and limiting degradative reactions. According to one aspect, the serum albumin may be a bovine serum albumin (BSA). In a further aspect, the saccharide may be a disaccharide such as sucrose or trehalose, or an oligosaccharide such as 2-hydroxypropyl-beta-cyclodextrin (CD).

According to one aspect, each of the one or more stabilizers may be loaded at a concentration of about 2 mg/mL to about 100 mg/mL. In some aspects, the amount of stabilizer(s) added may be based on the amount of urease present. In such aspects, the amount of stabilizer(s) added may be based on urease:serum albumin:saccaride molar ratios of about 1:5:1000 to about 1:20:10000. According to a particular aspect, the reagents layer 30 is further loaded with 2-hydroxypropyl-beta-cyclodextrin, sucrose, and bovine serum albumin in a ratio of about 1:12:3500:2000 of urease:bovine serum albumin:sucrose:2-hydroxypropyl-beta-cyclodextrin.

According to some embodiments, the bottom surface of the substrate of the reagents layer 30 of the test pad assembly 20, may be laminated to a top surface of the laminate layer 60 to isolate the reagents layer 30 from the adhesive 50 layer.

In some aspects, the laminate layer 30 may comprise a polymer or a copolymer such as a polyester, polyethylene terephthalate (PET), ethylene vinyl acetate (EVA), or any combination thereof. The adhesive layer 50 is engaged with the bottom surface of the laminate layer 60. It was discovered that certain adhesives may affect the pH of the reagents layer 30, which, in turn, may affect the pH indicator loaded therein, and thereby result in the occurrence of false positives or false negatives when analyzing salivary urea nitrogen (SUN) content of a sample. The laminate layer 60 isolates the reagents layer 30 from the adhesive layer 50, thereby negating any potential effects of the adhesive 50 on the physicochemical properties of the reagents layer 30.

In some aspects, the adhesive layer 50 may comprise one or more of epoxy adhesives, urethane adhesives, polyurethane adhesives, acrylic adhesives, and the like. In a particular aspect, the adhesive 50 is an acrylic adhesive.

In some embodiments, the test strip 10 may further comprise a control pad assembly 70 to facilitate the detection of azotemia or kidney disease. The control pad assembly 70 may be configured in the same manner as the test pad assembly 20 described above but without the addition of or loading of urease to the reagents layer 30. Thus, the control pad assembly 70 comprises a reagents layer 30 loaded with at least the pH indicator and the buffer but not with urease.

The control pad assembly 70 may therefore be used to provide a visual reference point when determining if a saliva sample has an increased concentration of urea nitrogen. In operation, a saliva sample may be applied to each of the test pad assembly 20 and the control pad assembly 70. The reagents layer 30 of the test pad assembly 20 is loaded with urease that will break down any urea present in the saliva sample to change the pH and thus the color of the test pad assembly 20. In contrast, the reagents layer 30 of the control pad assembly 70 does not include urease and thus will maintain the color of the initial pH and buffering capacity of the saliva sample. A user may then visually compare the color of the test pad assembly 20 to the color of the control pad assembly 70 to determine if a color change occurred on the test pad assembly 20 and thus conclude if the feline or canine has azotemia or kidney disease.

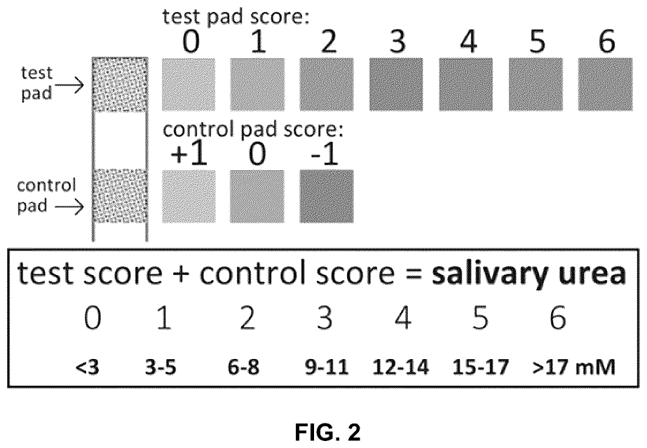

However, as will be appreciated, the initial pH of a saliva sample may vary between felines and canines as well as between different species of felines and canines. As a result, it may in some cases be difficult to distinguish between the color of the test pad assembly 20 and the color of the control pad assembly 70, depending on the initial pH of the saliva sample. Thus, a scoring system may be used to facilitate the determination of the salivary urea concentration. One example of such a scoring system is illustrated in FIG. 2. Using the illustrated scoring system, the salivary urea concentration may then be calculated as shown in Table 1 below:

TABLE 1
Calculation of Salivary Urea Concentration
test score + control score = salivary urea

| 0 | 1 | 2 | 3 | 4 | 5 | 6 |
|---|---|---|---|---|---|---|
| <3 | 3-5 | 6-8 | 9-11 | 12-14 | 15-17 | >17 mM |

Thus, by comparing the test pad assembly 20 to the control pad assembly 70, a user may determine whether a feline or canine has azotemia or kidney disease with a lower risk of false positives and false negatives.

In some embodiments, the color of the test pad assembly 20 and control pad assembly 70 may be measured using a spectrophotometer, a photometer, a CCD array on a smartphone camera, or the like.

As indicated above, the present disclosure also relates to a kit for detecting azotemia or kidney disease in felines and canines. In some embodiments, the present disclosure relates to a kit comprising one or more of the test strips 10 and instructions for use. In some aspects, the kit further comprises a reference card including, for example, the scoring system of FIG. 2 as well as the scoring calculation included above.

### EXAMPLES

### Example 1: Production of Test Pad Assemblies and Control Pad Assemblies

Two solutions were prepared for producing test pads and control pads according to Tables 2 and 3 below.

TABLE 2
Control Pad Solution

| | | | | Volume/Weight of | |
|---|---|---|---|---|---|
| | | | | Stock Required | |
| Ingredient | Stock Solution | Concentration | | for 10 mL | |
| Potassium | 1M, pH 5.60 | 30 | mM | 300 | mcL |
| Phosphate Buffer | | | | | |
| Bromothymol blue | 32 mM, 20% (v/v) | 2 | mM | 625 | mcL |
| | methanol, 87 mM | | | | |
| | NaOH | | | | |
| Tween 80 | 10% (v/v) | 0.05% (v/v) | | 50 | mcL |
| Poly(ethylene | Flakes, MW = | 50 | mg/mL | 500 | mg |
| glycol) | 3500-4500 | | | | |
| Bovine Serum | Lyophilized | 10 | mg/mL | 100 | mg |
| Albumin | powder | | | | |
| Sucrose | Granules | 15 | mg/mL | 150 | mg |
| (2-hydroxypropyl)- | Powder | 35 | mg/mL | 350 | mg |

beta-cyclodextrin

Phosphate buffer was added to 6 mL of distilled water, followed by bromothymol blue and tween 80. The remaining ingredients were then added one at a time into the solution and stirred until dissolved. The pH of the resultant solution was then adjusted to about 6.0 using a dilute hydrochloric acid solution and the volume topped off to 10 mL using distilled water.

Two sheets of Grade CF1 filter paper from Cytiva Health Sciences were each single-side laminated onto a sheet of GBC NAP-LAM® II film (NAP-LAM is a registered trademark of General Binding LLC, Zurich, IL, USA). Each of the laminated sheets were then immersed into one of the solutions to thereby load the filter paper with the solutions. The loaded laminated sheets were then dried at temperatures of about 40° C. to about 50° C. It is noted that the drying may also be completed using other techniques, such as freeze-drying. Each of the sheets was then adhered to a polystyrene carrier material using an acrylic adhesive and then finally cut into test pads and control pads.

TABLE 3
Test Pad Solution

| | | | | Volume/Weight of | |
|---|---|---|---|---|---|
| | | | | Stock Required | |
| Ingredient | Stock Solution | Concentration | | for 10 mL | |
| Potassium Phosphate | 1M, pH 5.60 | 40 | mM | 400 | mcL |
| Buffer | | | | | |
| Bromothymol blue | 32 mM, 20% (v/v) | 2 | mM | 625 | mcL |
| | methanol, 87 mM | | | | |
| | NaOH | | | | |
| Tween 80 | 10% (v/v) | 0.05% (v/v) | | 50 | mcL |
| Poly(ethylene glycol) | Flakes, MW = | 50 | mg/mL | 500 | mg |
| | 3500-4500 | | | | |
| Bovine Serum | Lyophilized | 10 | mg/mL | 100 | mg |
| Albumin | powder | | | | |
| Sucrose | Granules | 15 | mg/mL | 150 | mg |
| (2-hydroxypropyl)- | Powder | 35 | mg/mL | 350 | mg |
| beta-cyclodextrin | | | | | |
| Urease from | Lyophilized | 6 | mg/mL | 60 | mg |

| | | | |
|---|---|---|---|
| Canavalia ensiformis | powder | (539,880 IU/L) | (5,398.8 IU) |
| (Jack Bean) | 89,980 IU/g | | |

### Example 2: Testing Pad Assemblies in Felines and Canines

A pilot study to test the diagnostic performance of the test and control pads to detect high levels of blood urea nitrogen based on the salivary urea nitrogen, was carried out in three veterinary clinics.

The study was conducted as a cross-sectional survey of client owned canine and feline patients with a spectrum of health histories, including testing of healthy pre-operative and “wellness plan” patients, as well as sick patients with acute and chronic renal disease. A calculated population size of 346 canines and 116 felines was required to conduct a full scale clinical study with a power of 80% and to ensure that the 95% confidence intervals for the specificity and sensitivity are no wider than +/−10%, using an estimated disease prevalence rate of 10% for canines and 30% for felines over the age of 7. A trial population size of 92 canines and 56 felines were enrolled for a pilot study to get a preliminary understanding of the test's efficacy. Canine enrollment was limited to a required minimum of 4 disease positive cases and 25 disease negative cases, where disease positive is defined as a high or abnormal blood urea nitrogen level. Feline enrollment for each study site included a required minimum of 5 disease positive cases and 13 disease negative cases.

The test and control pads made in Example 1 were exposed to the saliva of various felines (n=56) and canines (n=92), including healthy, sick, and those with acute and chronic kidney disease, by rubbing the pads against the gums or under the tongue of the animals. All subjects were fasted for at least 2 hours prior to sample collection. After two minutes, the colors of the pads were visually compared to a color gradient and the concentration of saliva urea nitrogen was recorded. A blood sample was taken simultaneously or within 30 minutes of the saliva sample (and prior to any other treatments or fluid therapy) using standard collection techniques and an Adult Chem panel (CSA665; Antech Diagnostics Canada Ltd.) was used to compare the salivary urea nitrogen (SUN) and blood urea nitrogen (BUN) results.

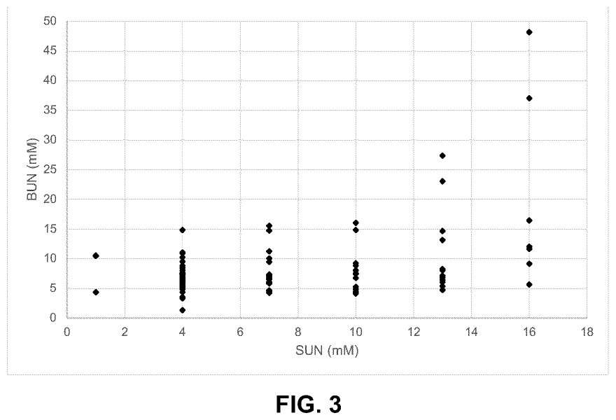

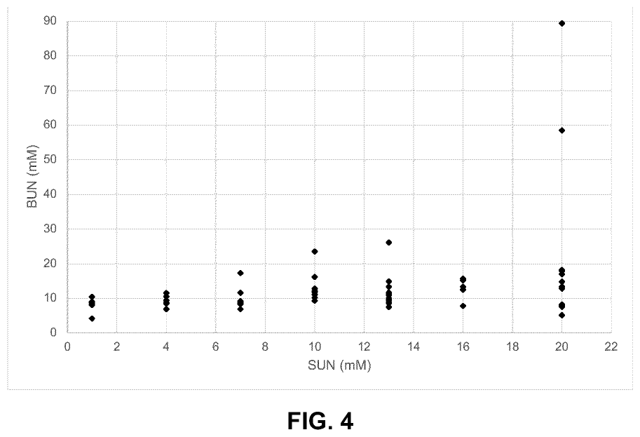

FIGS. 3 and 4 show the SUN vs. BUN results for canines and felines respectively. The BUN was tested at the same laboratory using the same method for all animals. Therefore, the laboratory cut-off for flagging abnormally high BUN (azotemia) was consistent for each species, ≥11.1 mM for canines and ≥12.9 mM for felines. The sensitivity (Sn) and specificity (Sp) was determined for each level of SUN found by the test strips and a Receiver Operating Characteristic (ROC) curve generated to describe the performance of the diagnostic to detect azotemia. As used herein, “sensitivity” refers to the ability of a test to detect disease-positive animals or the probability that a disease-positive animal will test positive. As used herein, “specificity” refers to the ability of a test to detect disease-negative animals or the probability that a disease-negative animal will test negative.

For a diagnostic screening test, the screening tool must have a sufficiently high degree of sensitivity (Sn) and a high negative predictive value (NPV), where a lower degree of specificity (Sp) and positive predictive value (PPV) can be tolerated. As can be noted in Table 4, choosing a lower SUN cut-off will result in a higher sensitivity, but will also result in significant false positives, i.e. at a SUN cut-off of ≥6-8 mM a sensitivity of 93% and specificity of 55% to detect BUN levels ≥11.1 mM. In one aspect, if the test showed a SUN level ≥6-8 mM then further investigation would need to be conducted such as blood and urine analysis to confirm azotemia. As those well versed in the use of medical tests will know, when using screening tests, one must be careful to direct the test towards a correct target population, otherwise significant over-testing and unnecessarily high false positives of a population can occur. In one aspect, it may be useful to direct this screening test towards geriatric canines and felines lacking clinical signs of disease and at risk of developing chronic kidney disease. In another aspect, it may be useful as a monitoring tool of kidney function for canines at risk of developing kidney disease from the use of non-steroidal anti-inflammatory drugs (NSAIDs), which tend to have significant negative effects on renal function in dogs.

Table 4 shows the sensitivity, specificity, positive predictive value (PPV) and negative predictive value (NPV) for each cut-off of SUN for canines. As used herein, “positive predictive value” refers to the probability that, given a positive test result, the animal is actually diseased. As used herein, “negative predictive value” refers to the probability that, given a negative test result, the animal is actually healthy.

TABLE 4
Canine Pilot Study Results

| | | | | | | True | True | False | False |
|---|---|---|---|---|---|---|---|---|---|
| | | Sn | Sp | PPV | NPV | Positive | Negative | Positive | Negative |
| SUN | ≥3-5 | 100% | 3% | 16% | 100% | 14 | 2 | 76 | 0 |
| Level | ≥6-8 | 93% | 55% | 27% | 98% | 13 | 43 | 35 | 1 |
| (mM) | ≥9-11 | 71% | 73% | 32% | 93% | 10 | 57 | 21 | 4 |
| | ≥12-14 | 57% | 85% | 40% | 92% | 8 | 66 | 12 | 6 |
| | ≥15-17 | 29% | 97% | 67% | 88% | 4 | 76 | 2 | 10 |
| | ≥17 | 0% | 100% | — | 85% | 0 | 78 | 0 | 14 |

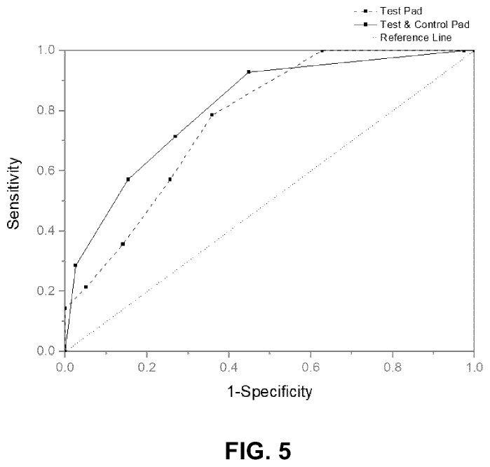

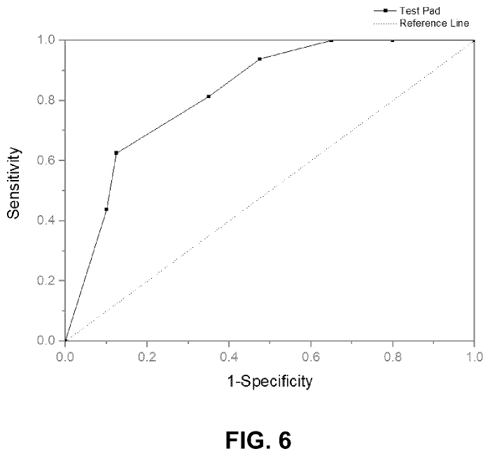

FIG. 5 is the ROC curve for diagnosing BUN ≥11.1 mM in canines using either the test pad alone or the test pad in conjunction with the control pad. FIG. 5 demonstrates the difference between diagnostic performance with and without incorporating the control pad to compensate for variable salivary pH. The area under the curve (AUC) is useful for comparing the diagnostic performance of different tests and is defined as the probability that a randomly selected, diseased animal has a greater test value than that of a randomly selected healthy animal. A perfect diagnostic test has an AUC=1. A slight increase in the AUC was found when incorporating the control pad, AUC=0.81 vs. AUC=0.77, and thus an increase in diagnostic performance is demonstrated when using a control pad with canine subjects. Table 5 and FIG. 6 show the results for felines. An AUC=0.83 was found for determining a BUN ≥12.9 mM using the salivary urea nitrogen test pads.

TABLE 5
Feline Pilot Study Results

| | | | | Likelihood | | Likelihood | | | | | |
|---|---|---|---|---|---|---|---|---|---|---|---|
| | | Sn | Sp | Ratio+ | PPV | Ratio− | NPV | True+ | True− | False+ | False− |
| SUN | ≥3-5 | 100% | 20% | 1.25 | 33% | 0.00 | 100% | 16 | 8 | 32 | 0 |
| Level | ≥6-8 | 100% | 35% | 1.54 | 38% | 0.00 | 100% | 16 | 14 | 26 | 0 |
| (mM) | ≥9-11 | 94% | 53% | 1.97 | 44% | 0.12 | 95% | 15 | 21 | 19 | 1 |
| | ≥12-14 | 81% | 65% | 2.32 | 48% | 0.29 | 90% | 13 | 26 | 14 | 3 |
| | ≥15-17 | 63% | 88% | 5.00 | 67% | 0.43 | 85% | 10 | 35 | 5 | 6 |
| | ≥17 | 44% | 90% | 4.38 | 64% | 0.63 | 80% | 7 | 36 | 4 | 9 |

It is clear from this data that the test and control pads may be used to screen for high blood urea nitrogen in a large, cross-sectional sample size of both feline and canine patients.

### Example 3: Relationship Between Salivary Urea Concentration and Hue

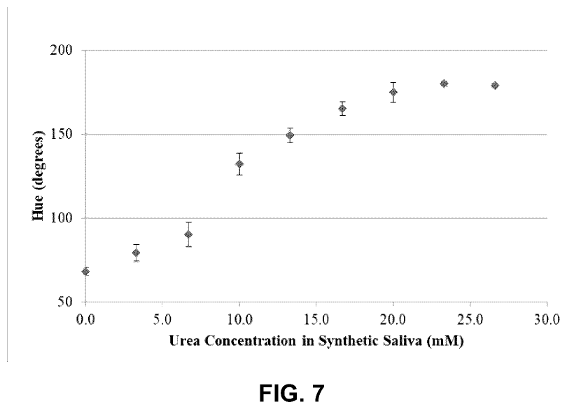

Test pads were made using the method disclosed in Example 1. The test pads were then exposed to synthetic cat saliva having an initial pH of 8.6 spiked with 0, 3.3, 6.7, 10.0, 13.3, 16.7, 20.0, 23.3, or 26.7 mmol/L of urea. The hue of the test pads was subsequently recorded using a smartphone CCD array camera (iPhone 6), the results of which are outlined in FIG. 7.

In more detail, as shown in FIG. 7, the hue of the tests pads increases with the urea concentration of the saliva sample, thereby illustrating the detection capabilities of the test strips of the present disclosure. It is noted that the recorded hue values correspond to a change in color from yellow (lowest urea concentration) to dark blue (highest urea concentration).

### Example 4: Shelf Life of Test Strips

Three batches of test pads were produced using the method outlined in Example 1 but with varying stabilizers:
I. 10 mg/mL of Bovine serum albumin (BSA) (molar ratio of 12, urease:BSA);
II. 15 mg/mL of sucrose and 35 mg/mL of 2-hydroxypropyl)-beta-cyclodextrin (CD) (molar ratio of 1:3504:2032, urease:sucrose:CD); and
III. 10 mg/mL of BSA and 15 mg/mL of sucrose and 35 mg/mL of (2-hydroxypropyl)-beta-cyclodextrin (molar ratio of 1:12:3504:2032, urease:BSA:sucrose:CD).

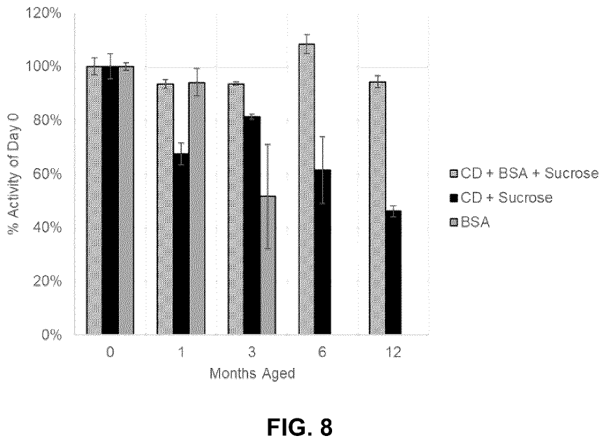

The produced test pads were then stored in a dark environment in sealed HDPE bottles in temperatures from a range between 18° C. and 24° C. and humidity from a range of 35% to 45%. The stored test pads were tested at a number of time points using synthetic cat saliva spiked with 13.3 mmol/L of urea to determine the percent remaining urease activity of each set of test pads, as measured by the hue of the test pads after exposure to the synthetic cat saliva and incubation for 2 minutes. The results of the testing are illustrated in FIG. 8.

As shown by FIG. 8, the combination of BSA, sucrose, and (2-hydroxypropyl)-beta-cyclodextrin represents the most effective combination of stabilizers. Regardless, it is clear that any combination of stabilizers may provide the test pads of the test strips of the present disclosure with a shelf life that is considerably longer than that of the conventional test strips developed for use with human biological samples.

1. A test strip for detecting azotemia or kidney disease in felines and canines comprising:
an elongate carrier material; and
a test pad assembly adherable to the elongate carrier material, the test pad assembly comprising a reagents substrate material loaded with urease, a pH indicator with a transition interval of pH 5 to pH 9, and a buffer for adjusting a pH value of a feline or canine saliva sample to within the transition interval of the pH indicator.

2. The test strip according to claim 1, wherein the test pad assembly is laminated on a side adhered to the elongate carrier material.

3. The test strip according to claim 2, wherein the test pad assembly is laminated with a polyester, a polyethylene terephthalate (PET), an ethylene vinyl acetate (EVA), or any combination thereof.

4. The test strip according to claim 1, wherein the test pad assembly is adhered to the elongate carrier material using an epoxy adhesive, a urethane adhesive, a polyurethane adhesive, an acrylic adhesive, or any combination thereof.

5. The test strip according to claim 1, further comprising a control pad assembly comprising the reagents substrate material loaded with the pH indicator and the buffer.

6. The test strip according to claim 5, wherein the control pad assembly is adhered to the elongate carrier material in a selected location adjacent to the location of the adhered test pad assembly.

7. The test strip according to claim 1, wherein the reagents substrate material comprises cellulose, nitrocellulose, cellulose acetate, a polyester, a nylon, or any combination thereof.

8. The test strip according to claim 1, wherein the reagents substrate material is impregnated with the urease, the pH indicator, and the buffer.

9. The test strip according to claim 1, wherein the reagents substrate material has a water absorptivity of about 15 μL/cm² to about 30 μL/cm².

10. The test strip according to claim 1, wherein the pH indicator indicates pH changes over a pH range of about 6 to about 8.

11. The test strip according to claim 1, wherein the pH indicator comprises phenol red, bromothymol blue, bromocresol purple, methyl red, or a combination thereof.

12. The test strip according to claim 1, wherein the buffer comprises sodium phosphate, potassium phosphate, 2-[Bis(2-hydroxyethyl)amino]-2-(hydroxymethyl)propane-1,3-diol, 2,2′-(Propane-1,3-diyldiimino)bis[2-hydroxymethyl)propane-1,3-diol], or a combination thereof.

13. The test strip according to claim 1, wherein the reagents substrate material is further loaded with one or more wetting agents comprising a polyethylene glycol, a polysorbate, or a combination thereof.

14. The test strip according to claim 1, wherein the reagents substrate material is further loaded with one or more stabilizers comprising a serum albumin, a saccharide, or a combination thereof.

15. The test strip according to claim 14, wherein the one or more stabilizers comprise bovine serum albumin, sucrose, 2-hydroxypropyl-beta-cyclodextrin, or a combination thereof.

16. The test strip according to claim 14, wherein the reagents substrate material is further loaded with the one or more stabilizers at a urease:serum albumin:saccaride molar ratio of about 1:5:1000 to about 1:20:10000.

17. A kit comprising a plurality of the test strip according to claim 1 and instructions for use.
